(19) United States
(12) Patent Application Publication
(10) Pub. No.: US 2025/0186768 A1
(43) Pub. Date: Jun. 12, 2025

(54) BIOELECTRONIC MODULATION OF NERVE-CANCER COMMUNICATION TO INFLUENCE THE TUMOR MICROENVIRONMENT

(71) Applicant: Scuola Superiore di Studi Universitari e di Perfezionamento Sant'Anna, Pisa PI (IT)

(72) Inventors: Eugenio REDOLFI RIVA, Pisa (IT); Ciro ZINNO, Portici (IT); Debora ANGELONI, San Giuliano Terme (IT); Silvestro MICERA, Firenze (IT)

(73) Assignee: Scuola Superiore di Studi Universitari e di Perfezionamento Sant'Anna, Pisa PI (IT)

(21) Appl. No.: 18/537,387

(22) Filed: Dec. 12, 2023

(51) Int. Cl. A61N 1/36; A61N 1/05

(52) U.S. Cl. CPC A61N 1/36002; A61N 1/0504; A61N 1/36135; A61N 1/36175; A61N 1/36192; A61N 1/36196

(57) ABSTRACT

The invention relates to a method for the treatment of cancer or adjuvating the treatment of cancer by exploiting the bidirectional tumor-nerve communication as novel pathway to induce an anticancer activity towards epithelial cancer cells using bioelectronic neuromodulation aimed at influencing the TME status.

### FIELD OF THE INVENTION

The invention relates to a method for the treatment of cancer or augmenting the treatment of cancer by exploiting the bidirectional tumor-nerve communication as novel pathway to induce an anticancer activity towards epithelial cancer cells using bioelectronic neuromodulation aimed at influencing the tumor microenvironment (TME) status.

### BACKGROUND OF THE INVENTION

Cancer is the second cause of death worldwide and represents a significative burden for society, leading to numerous deaths caused by late diagnosis, chemoresistance and drug therapies' side effects. Drugs inefficacy do indeed represent the bottleneck of actual cancer therapies. Chemotherapeutics need to be systemically delivered at high doses to reduce primary tumor size and metastases, with frequent detrimental effects for patients' organs, including myelodepression, cardiac and hepatic toxicity and eventually secondary mutagenic effects. In addition, chemoresistance to currently available treatments frequently occur, leading to tumor recurrence in a site that is not necessarily identical to the primary one. In some cases, the side effects of the drug therapies are so devastating that the drug treatment must be stopped and switched to palliative treatment to avoid suffering. Moreover, drug action is only controllable by the timing of administration and dose and fail to solve the inefficacy to distinguish cancer cells from the healthy ones. Monoclonal antibodies address in part this issue but cause adverse immune reactions with severe side effects and can be used only if the cancer cells express target receptors that the drug can bind to exploit its action. Recently, immunotherapy is establishing as novel promising cancer treatment that can restore the immune cells action against the tumor. However, as for monoclonal antibodies, its action is cell-specific and unfortunately few cancer types can nowadays be treated using this approach.

Multidisciplinary approach could pave the way to innovative therapeutic strategies. In recent years, the understanding of tumor microenvironment (TME) properties allowed a better comprehension of tumor progression modalities. TME is the very first niche in which cancer cells start to grow and is composed by blood vessels, nerves, stromal and immune cells that surround the cancerous masses and acts as crucial player in cancer growth and metastasis formation. To proliferate and seed secondary organs, cancer cells modulate (and are modulated by) their TME components in a pro-tumoral way, triggering blood vessels formation via angiogenesis and providing nutrient supply to support cell replication. Concomitantly, tumor masses must evade immunosurveillance to grow and thrive. Cancer cells escape immunity by dysregulating immune checkpoint signaling and by attracting immunosuppressive cells within the TME, such as myeloid-derived suppressor cells (MDSCs). Nerve fibers formation and spreading within the tumor niche have been recently discovered to alter TME properties via neurotransmitters release. Cancer drives neurogenesis to modulate the TME in a pro-tumoral fashion, enhancing cancer growth, angiogenesis, immunosurveillance escape and favoring metastasis.

Therefore, targeting TME state and pathways indeed represents an innovative and promising solution against cancer spreading.

A particular TME component is attracting substantial interest for its newly discovered role in modulating cancer growth and metastatic spreading: the intermingled network of intratumoral nerve fibers. Histological studies revealed the presence of afferent and efferent nerve fibers (autonomic and sensorial) within the TME of main epithelial tumors (breast, prostate, pancreatic, ovarian, lung cancers and melanoma), often correlated with poor prognosis. Nerves and TME establish a bidirectional communication that fuels cancer growth. Adrenergic signaling possesses a direct effect on cancer cell proliferation, as demonstrated with surgical denervation in a mouse model of prostate carcinoma. β-adrenergic receptors have shown to be involved in tumor growth and immunosurvelliance escape, as they are expressed on both cancer and immune cells. Catecholamines (CA) release within the TME by nerves fibers triggers angiogenesis and has a direct effect on tumor growth and spreading by acting on via cAMP pathway upregulation upon β-adrenergic receptors binding; concomitantly, the density of sympathetic (SN) and parasympathetic (PSN) nerve fibers within the TME is associated with poor clinical outcomes. Recent studies showed high dependence of cancer cell types on the amount and nature of tumor innervation within the TME. Interestingly, SN and PSN innervation, and consequent release of neurotransmitters within the TME, triggers a feedback effect capable of modulating cancer growth and immunosurveillance in a murine breast cancer model, with both SN and PSN displaying opposite effects on increasing and hindering tumor growth, respectively. This study also elucidated the importance of neurotransmitters released within the TME to modulate immunosurveillance, highlighting the crucial importance of the interaction between these three components (nerves—cancer cells—TME) on regulating cancer growth and metastatic spreading. Specifically, CA favors suppression of the cytotoxic function of CD8+ lymphocytes and natural killer cells (NK), that play a crucial role in cancer immunosurveillance. On the contrary, PN decreased the expression of PD-L1 on tumor tissues and restored immunosurveillance by increasing expression of INFγ on CD4+ and CD8+ lymphocytes, supporting the hypothesis that cancer innervation can modulate TME components in both pro- and anti-tumorigenic fashion depending on the cancer model.

Tumor innervation, especially SN nerve fibers innervating the TME, have recently been the target of a pharmaceutical approach, based to the delivery of antagonist drugs (β-blockers) that inhibit the β-adrenergic receptors expressed by cancer cells, immunomodulatory drugs, local anesthetics, and nerve blockers molecules. Several clinical trials of this class of drugs are currently under evaluation for its potential to reduce cancer growth. However, this approach presents systemic anticancer drug therapies, which have significant disadvantages and limitations such as long-term organ toxicity, myelosuppression, chemoresistance and above all lack selectivity towards the target tumor cell. Repeated systemic infusion of the active principle is the only way to tune the drug dose, which however does not allow precise and targeted treatment towards the target lesion.

The technical problem faced and solved by the present invention is to address the lack of efficacy and selectivity of current anticancer therapies using a completely new state-of-the-art approach.

### SUMMARY OF THE INVENTION

The solution proposed by this invention is to exploit the bidirectional tumor-nerve communication as novel pathway to influence the TME status in order to induce an anticancer activity towards epithelial cancer cells using bioelectronic neuromodulation.

Bioelectronic Medicine (BM) holds the potential to modulate physiological functions by implanting a Neural Interface (NIs) on defined target nerves and stimulating/inhibiting its activity. It aims to treat several diseases (such as obesity, diabetes, autoimmune diseases, psychiatric and neurodegenerative disorders) by electrically modulating the activity of the peripheral nerve linked to the target organ. Low-invasive modulation is possible with extraneural NIs, that are wrapped around the target nerve, preserving its integrity. Selective fascicle modulation is possible by intrafascicular NIs implantation, that allow penetrating the nerve with the electrode. Neuromodulation can indeed stimulate the action potential of a target nerve but may also be used to inhibit such signal. In this regard, action potential blockage kilohertz frequency alternating current (KHFAC) or low-frequency stimulation can be performed.

The present invention relates therefore to a method of treating a tumor, wherein the imaging techniques, as histological and immunohistochemical analysis of the target nerve, are fundamental to be able to target the proper structures. In the framework of the present invention, obtaining an innervation map of the target tumor is of crucial importance, since they aim to select an anatomically relevant cancer nerve trunk, in order to implant a Neural Interface (NI) on it and link it to an external stimulator for the control of a neuromodulation protocol.

Moreover, a graphical user interface (GUI, such as a laptop or tablet) is connected to a digital/analog converter that allows to tune the stimulator's electrical parameter for neuromodulation experiments (amplitude, frequency, pulse width and timing of the stimulation).

To study the effect of stimulation or inhibition of the action potential of a nerve that innervates the TME, electrical parameters for stimulation/inhibition are defined during training experiments by varying several experimental conditions. Training experiments performed in animal models are also used to study the kinetic of tumor growth and spreading, to investigate the dynamics of primary tumor mass growth, the evolution of its TME, the innervation modality and the tumor spreading, analyzing the timing that it requires to invade secondary organs and the innervation of metastasis. This information is used to plan the neuromodulation protocol.

The present invention also relates to a neuromodulation protocol imposed to reduce cancer growth and spreading. The same paradigm is used to target the primary tumor mass as well as secondary metastatic sites, as they are the major responsible for patient death. As the communication between tumor and nerves has reported to be bidirectional, the NIs are also used to acquire information regarding the tumor status, by recording the action potential that travels to the TME using the very same NI used for the bioelectronic modulation of TME.

### Advantages

The solution proposed by this invention represents a completely novel paradigm of selective cancer therapy that could drive breakthrough innovation in not only BM and TME analysis, but also in Clinical Oncology.

Several beneficial applications of BM have already been shown with remarkable positive impacts on patients' quality of life. For this reason, the technological solution proposed by the present invention has the potential to pave the way for a completely new way of treating cancer.

The solution proposed by the present invention is expected to have a significant societal and industrial impact. Bioelectronic modulation has already been demonstrated to significantly improve patient's quality of life as previously described. Translating this approach in the oncology field could allow a completely new line of anticancer treatment, characterized by high-precision and controllability. Contrary to anti-cancer drug therapies, whose efficacy strictly depends on the cell targeting capability of the therapeutic molecule, bioelectronic approaches indeed possess the favorable above-mentioned features.

However, this approach does not intend to replace drug therapies. In fact, by modulating TME in an anti-oncogenic direction, possible improvement of drug anticancer efficacy could be expected, envisioning the bioelectronic approach as an adjuvant therapy to the current anti-cancer protocols, in order to improve patients' quality of life and overall survival.

Hence, object of the present invention is a method for the treatment of a cancer in a subject, or for adjuvating, i.e. enhancing or augmenting the treatment of cancer in a subject comprising the following steps:
i) implanting a neural interface (NI) on at least one target nerve of the TME of a tumor mass of said cancer, and at least one stimulator in the subject; and
ii) performing a neuromodulation protocol to said nerve using said neural interface in order to reduce the tumor mass or the metabolic activity of said cancer.

In each part of the present description, the term “comprising” can be replaced by the term “consisting of”.

Further advantages and/or embodiments of the present invention will be evident from the following detailed description.

### DETAILED DESCRIPTION OF THE FIGURES

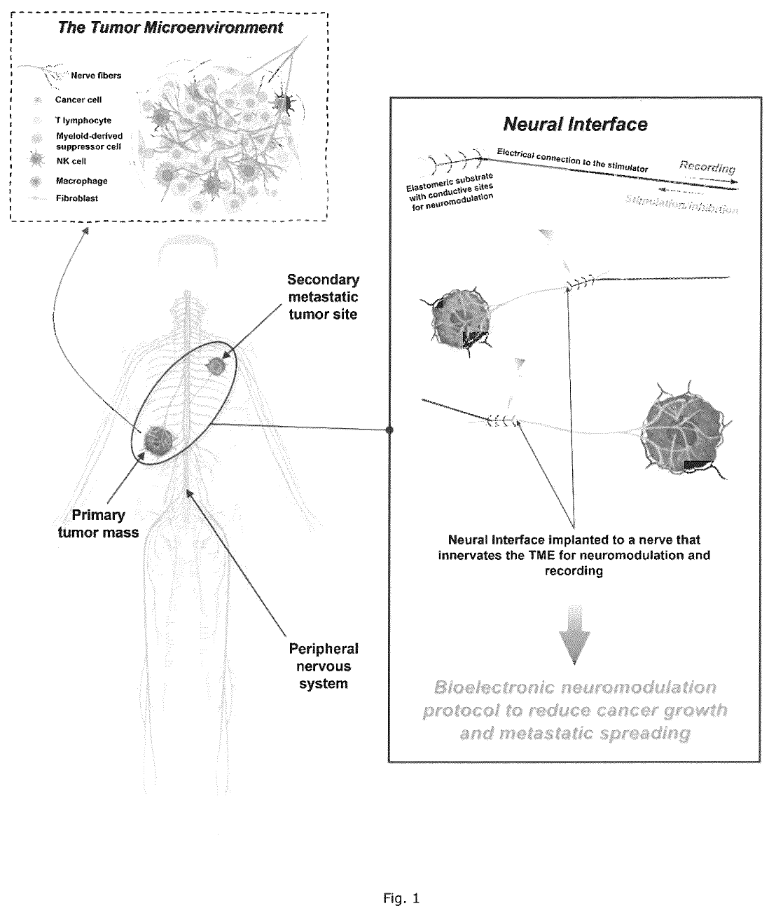

FIG. 1 depicts the Concept of Bioelectronic modulation of the cancer innervation; and

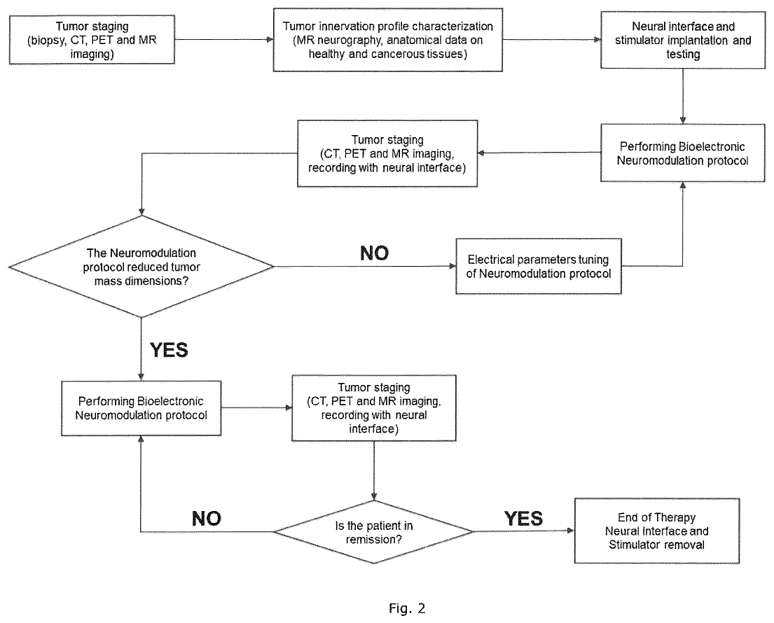

FIG. 2 depicts one preferred embodiment of the present invention, in particular the Workflow of the method according to one embodiment of the invention is schematized.

### GLOSSARY

Before the methods of the present disclosure are described in greater detail, it is to be understood that the methods are not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods. The upper and lower limits of these smaller ranges may independently be included in the smaller ranges and are also encompassed within the methods, subject to any specifically excluded limit in the stated range. Where the stated range includes one or both of the limits, ranges excluding either or both of those included limits are also included in the methods.

Certain ranges may be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

For purposes of the present invention, the term “adjuvating” is used to describe the process of augmenting, potentiating or otherwise improving the therapeutic benefits of a primary cancer therapy. Sometimes referred to as “adjuvant therapy” or adjunct therapy or augmentation therapy, an adjuvating therapy is a therapy is given in addition to a primary therapy to manage the effectiveness of a primary therapy.

According to the present invention, the expression “adjuvating the treatment of a cancer in a subject” shall be understood to include utilizing additional substances or therapies to enhance the effectiveness of the primary cancer treatment that a patient is undergoing. Adjuvants in cancer therapy are often used to boost the body's immune response, improve the efficacy of chemotherapy or radiation, or support other primary treatment modalities. The goal is to improve the overall outcome of cancer treatment for the individual patient.

According to the present invention, the term “target nerve” refers to any nerve that innervates the tumor microenvironment (TME), in particular it refers to any nerve coupled with the tumor mass, metastasis, or the tissue surrounding the tumor mass.

According to the present invention, the term “tumor microenvironment or TME” refers to the dynamic and intricate ecosystem surrounding a tumor, encompassing a diverse array of cellular and non-cellular elements, including cancer cells, immune cells, fibroblasts, blood vessels, extracellular matrix, and signaling molecules.

According to the present invention, the term ‘Tumor Mass’ relates to the physical accumulation of neoplastic cells within a localized area, forming a discernible and abnormal tissue structure. The Tumor Mass may comprise a heterogeneous population of cells, including but not limited to cancer cells, stromal cells, and immune cells.

According to the present invention, the term “metabolic activity” relates to cell metabolism.

According to the present invention, the term “parasympathetic, sympathetic and sensory innervation of the tumor microenvironment (TME)” means the regulation of neural signals within the TME by the parasympathetic, sympathetic, and sensory branches of the autonomic nervous system, respectively.

According to the present invention, the term “glucose consumption of said cancer” means the rate at which the cancer cells use glucose for metabolic processes, providing an indication of the metabolic state of the cancer.

According to the present invention, the term “biphasic rectangular pulses” means electrical pulses characterized by two phases of opposite polarity, each having a distinct duration, facilitating controlled nerve stimulation.

According to the present invention, the term “biphasic sinusoidal pulses” means a type of electrical pulses characterized by a waveform that resembles a sinusoidal pattern and has two phases. “Biphasic” indicates that the pulse has both positive and negative phases.

According to the present invention, the term “biphasic trapezoidal pulses” means a type of electrical pulses with a waveform resembling a trapezoid and having two phases. The “biphasic” nature implies that the pulse comprises both positive and negative phases.

According to the present invention, the term “biphasic triangular pulses” means electrical pulses characterized by a triangular waveform and having two phases. Similar to the previous terms, “biphasic” indicates the presence of both positive and negative phases in the pulse.

According to the present invention, the term “amplitude” of the pulse means the magnitude of the electrical current in the stimulation pulse.

According to the present invention, the term “frequency” of the pulse means the number of pulses delivered per unit of time, determining the rate at which neural stimulation occurs.

According to the present invention, the term “pulse width” means the duration of each individual pulse in a stimulation set.

According to the present invention, the term “monopolar”, “bipolar stimulation” or “tripolar stimulation” means the application of electrical stimulation using either a single electrode, two electrodes or three electrodes with opposite polarities, respectively.

According to the present invention, the term “biphasic sinusoidal waveform” means an electrical waveform characterized by alternating positive and negative phases, imparting specific temporal and amplitude characteristics to the neural stimulation.

According to the present invention, the term “biphasic square waveform” means an electrical waveform that has a square shape and consists of two phases. The “biphasic” nature indicates that the waveform includes both positive and negative phases.

According to the present invention, the term “biphasic trapezoidal waveform” means an electrical waveform with a trapezoidal shape and two phases. Similar to the square waveform, “biphasic” implies the presence of both positive and negative phases in the waveform.

According to the present invention, the term “biphasic triangular waveform” means an electrical waveform with a triangular shape and two phases. As before, “biphasic” indicates that the waveform includes both positive and negative phases.

According to the present invention, the expression “said neural interface (NI) is placed around the target nerve” means the physical positioning of the neural interface in proximity to the specified nerve to facilitate effective and targeted electrical stimulation.

According to the present invention, the term “current-controlled stimulator” means a stimulator device wherein the delivered electrical current is precisely regulated, allowing for controlled and tailored neural stimulation.

According to the present invention, the term “spike count” means the total number of action potentials or spikes generated by a neuron within a specified time period. It quantifies the frequency of neural activity and may be utilized to assess the level of excitation or inhibition in a neuronal network.

According to the present invention, the term “evoked compound action potential shape or area” means the characteristic waveform or total area under the curve of the compound action potential (CAP) generated in response to a specific external stimulus. This term may describe the shape, amplitude, and temporal characteristics of the evoked neural response, providing valuable information about the functionality of neural pathways.

According to the present invention, the term “firing rate” means the rate at which a neuron generates action potentials over time. It represents the frequency of neural firing and is a crucial parameter for understanding the dynamics of neural networks. Firing rate is often measured in spikes per second (Hz) and can be indicative of the neuron's activity level or responsiveness to stimuli.

According to the present invention, the term “spike shape” means the characteristic waveform or morphology of an individual action potential or spike generated by a neuron. It includes features such as amplitude, duration, and shape, providing insight into the unique electrical signature of the neuron's firing pattern. Spike shape analysis can be essential for distinguishing different types of neurons or assessing the impact of experimental interventions on neural activity.

### DETAILED DESCRIPTION OF THE INVENTION

Unless defined otherwise, all technical and scientific terms used herein have the same meaning as commonly understood by one of ordinary skill in the art to which the methods belong. Although any methods similar or equivalent to those described herein can also be used in the practice or testing of the methods, representative illustrative methods and materials are now described.

It is appreciated that certain features of the methods, which are, for clarity, described in the context of separate embodiments, may also be provided in combination in a single embodiment. Conversely, various features of the methods, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed, to the extent that such combinations embrace operable processes and/or devices/systems/kits. In addition, all sub-combinations listed in the embodiments describing such variables are also specifically embraced by the present methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order which is logically possible.

According to a first aspect, there is provided a method for the treatment of a cancer in a subject, or for adjuvating the treatment of cancer in a subject comprising the following steps:
i) implanting a neural interface (NI) on at least one target nerve of the tumor microenvironment (TME) of a tumor mass of said cancer, and at least one stimulator connected to said NI in the subject requiring the treatment; and
ii) performing a neuromodulation protocol to said nerve using said neural interface in order to reduce the tumor mass or the metabolic activity of said cancer.

By “treatment”, it is meant at least an amelioration of the symptoms associated with the disease condition afflicting the subject (i.e., host), where amelioration is used in a broad sense to refer to at least a reduction in the magnitude of a parameter, e.g., symptom, associated with the pathological condition being treated, such as size of tumor, rate of growth of tumor, spread of tumor, pain, etc. As such, treatment also includes situations where the pathological condition, or at least symptoms associated therewith, are completely inhibited, e.g., prevented from happening, or stopped, e.g., terminated, such that the host no longer suffers from the pathological condition, or at least the symptoms that characterize the pathological condition. Preferably where the symptom being treated is pain, treatment in accordance with methods of the invention results in some instances in a decrease in the National Initiative on Pain Control (NIPC) numerical scale of 1 point or more, such as 2 points or more, 3 points or more, 4 points or more, 5 points or more, 6 points or more, 7 points or more, 8 points or more, including 9 points or more. As such, treatment includes both curing and managing a pain condition. Where the symptom being treated is tumor growth, treatment in accordance with methods of the invention results in some instances in a decrease in the rate of tumor growth, e.g., as compared to a suitable control, where the magnitude of the decrease in rate may be 5% or greater, such as 10% or greater, including 20% or greater. In some instances, treatment in accordance with methods of the invention results in a reduction in tumor size, where the reduction may be 5% or more, including 10% or more, such as 15% or more, e.g., 25% or more, 50% or more, 75% or more, v/v.

A variety of subjects are treatable according to the methods of the invention. Subjects treatable as described herein include “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In preferred embodiments, the subject is human.

In one embodiment, the stimulator is implanted in a subcutaneous cavity.

In one embodiment, the stimulator is connected to the NI by means any connecting mean known by the skilled person, preferably by means of encapsulated lead wires to the NI.

In one embodiment, the neuromodulation protocol is performed in order to stimulate or inhibit said target nerve.

The neuromodulation protocol is carried out in two different steps: i) establishment of a stimulation/inhibition protocol by characterization of the parasympathetic, sympathetic and/or sensory innervation of the tumor and ii) selection of the neuromodulation parameters according to the stimulation/inhibition protocol. In particular, the following ranges will be used for stimulation: biphasic rectangular pulses with amplitude 1-10 mA, frequency 1-100 Hz, pulse width 0.1-3 ms. For the inhibition protocol, the following ranges will be applied: biphasic sinusoidal waveforms, amplitude 1-5 mA, frequency 1-100 kHz, pulse width 0.01-1 ms. The neuromodulation protocol will be possibly modified during the therapy according to the results observed in terms of reduction of tumor mass or the metabolic activity of said cancer.

The present invention aims therefore at overcome the bottleneck of current drug protocols: the lack of selectivity towards the cancerous cells. By targeting a nerve branch that innervates the TME of the primary or metastatic tumor, the therapeutic action is directed exclusively towards the component located downstream of the target nerve fibers: the TME. Conversely, β-blockers and other drugs' action fails to discriminate between the fibers that innervate the tumor mass and other fibers that innervate vital organs outside the tumor, therefore running the risk of interfering with their physiological function. Targeting cancer growth using Bioelectronic modulation of tumor-nerve communication not only allows for a precise and selective therapeutic action towards the cancerous mass, but also enables a highly controllable therapeutic action, whose action can be modulated in real-time simply by acting on the electrical parameters of the electrical stimulation or inhibition. Furthermore, this therapeutic intervention can be easily interrupted by acting on the stimulator connected to the NI and can also record the nerve signal in the target nerve, by reading its signal instead of electrically modulating it. This occurrence would be useful to acquire information on the cancerous mass, using them as feedback to control and tune the neuromodulation parameters.

In one embodiment, the target nerve is selected by characterization of the innervation of the tumor microenvironment (TME), in particular for primary and metastatic tumor masses of said cancer. More in particular, the method may include collecting physiologic data, electrophysiological data, neural traffic, sympathetic neural traffic, parasympathetic neural traffic, sensory neural traffic, afferent neural traffic, efferent neural traffic, smooth muscle response, or the like from the target organ and/or within the vicinity of the target organ. Such information may be advantageous for determining the extent of a treatment, a disease state of the organ, for predicting the response of the organ and/or a neural circuit connected thereto to a treatment, an ablation, a delivery of energy, or the like. The target nerve can be identified by combining imaging information, such as MRI, CT and ultrasound scans, with physiological and/or neural information. The target nerve can be (not limited to) a major autonomic nerve, e.g., vagus nerve, splanchnic nerve, thoracic nerves, innervating the TME, with a characteristic dimension suitable to implant a NI. Characteristic dimension suitable to implant a NI ranges from 200 μm to 5 mm. In one embodiment, multiple neural interfaces are implanted in the target nerve, to influence TME and affect cancer growth.

In one embodiment, the method for the treatment of a cancer in a subject, or for adjuvating the treatment of cancer in a subject of the present invention comprises the following steps:
i) acquiring data about the staging of the cancer of said subject;
ii) defining the tumor microenvironment (TME) innervation profile of each tumor mass of said cancer;
iii) implanting a neural interface (NI) on at least one target nerve of the tumor microenvironment (TME) of a tumor mass of said cancer, and at least one stimulator; and
iv) performing a neuromodulation protocol using said neural interface in order to reduce the tumor mass or the metabolic activity of said cancer.

In one embodiment, the method for the treatment of a cancer in a subject, or for adjuvating the treatment of cancer in a subject according to the present invention, comprises the following steps:
i) acquiring data about the staging of the cancer of said subject before the treatment;
ii) defining the tumor microenvironment (TME) innervation profile of each tumor mass of said cancer;
iii) implanting a neural interface (NI) on at least one target nerve of the tumor microenvironment (TME) of a tumor mass of said cancer, and at least one stimulator
iv) performing a neuromodulation protocol using said neural interface with neuromodulation parameters related to the tumor staging before the treatment;
v) comparing data about the staging of the cancer of said subject before and after the treatment; and
vi) a) if no reduction of said tumor mass or the metabolic activity has been achieved, repeat the step iv) changing the neuromodulation parameters; or
   b) if reduction of said tumor mass or the metabolic activity has been achieved, repeat step iv) without changing the neuromodulation parameters. In one embodiment said neuromodulation protocol comprises the selection of the following parameters amplitude, frequency, pulse width, duration of single stimulation and number of daily sessions.

In one embodiment said neuromodulation protocol comprises the selection of the following parameters amplitude, frequency, pulse width, duration of single stimulation and number of daily sessions. Amplitude, frequency and pulse width can be modified in order to recruit nerve fibers of different type and size according to the target nerve. For example, increasing the amplitude allows to recruit more nerve fascicles and fibers, while increasing the pulse width allows to recruit nerve fibers with smaller diameter. The modification can be aimed at improving the effects of the therapy and decreasing the possible adaptation that the patient could undergo. Moreover, modification of amplitude, frequency and pulse width allows modifying the sensation given by the stimulation, such as tingling and slight muscle contraction. The duration and the number of sessions for the neuromodulation protocol will be established at the start of the therapy and then modified according to the efficacy of the therapy itself and the feedback given by the patients.

In one embodiment, the method for the treatment of a cancer in a subject, or for adjuvating the treatment of cancer in a subject according to the present invention, comprises the following steps:
i) acquiring data about the staging of the cancer of said subject before the treatment;
ii) defining the tumor microenvironment (TME) innervation profile of each tumor mass of said cancer;
iii) implanting a neural interface (NI) on all the nerves of the tumor microenvironment (TME) of each tumor mass of said cancer, and their stimulators; and
iv) performing a neuromodulation protocol using said neural interface in order to reduce the tumor mass or the metabolic activity of said cancer.

Reduction of tumor mass is the main goal of the proposed invention and can be performed in several ways with the methods according to the present invention, depending on the TME status of the said epithelial tumor, on the effect of the neuromodulation protocol on the said TME status, and on the nature of the TME innervation where the neural interface (NI) is implanted.

The neuromodulation protocol according to the present invention aims to modify the neurotransmitters concentration within the TME. Tumor cells, immune cells, and stromal cells within TME possess receptors that binds said neurotransmitters and their action can be therefore influenced by said neuromodulation protocol. As a non-limiting example, tumor mass reduction is achieved upon neuromodulation thanks to the restoration of the immunosurveillance within the TME, due to the effect of the neuromodulation protocol on the immune cell population.

Another effect that reduces tumor mass size is the alteration of another component of tumor TME: the tumor mass vascularization. By carrying out the neuromodulation protocol according to the present invention, the neovascularization of tumor mass is reduced, which is correlated to a reduction of tumor growth.

Moreover, the neuromodulation protocol according to the present invention has a direct effect on tumor cells within the TME, as cancer cells possess cell receptors that binds to neurotransmitters released directly within the TME, thanks to said neuromodulation protocol.

Therefore, tumor mass reduction, is defined as dimensional decrease of the initial status of said tumor mass, evidenced during staging by imaging techniques, such as CT, PET, and MRI scan.

However, a positive effect of the neuromodulation protocol according to the present invention is not only related to dimensional reduction, but also to a lower cancer cells metabolic activity. In this regard, PET imaging provides information on the glucose consumption by cancer cell, which is correlated to their viability. A significative reduction in glucose consumption is a positive index that is linked to a reduction of cancer cell viability (even without a significative dimensional reduction of tumor cell) and must be interpreted as an evidence of the effectiveness of the neuromodulation protocol.

In one embodiment the neuromodulation protocol uses the following parameters for stimulating the target nerve: biphasic rectangular, sinusoidal, trapezoidal, triangular pulses or a combination thereof, with:
an amplitude in a range from 1 to 10 mA; and/or
a frequency in a range from 1 to 100 Hz;
pulse width is in a range from 0.01 to 3 ms; and
stimulation is a monopolar, bipolar or tripolar stimulation.

In one embodiment, the neuromodulation protocol uses the following parameters for inhibiting the target nerve: biphasic sinusoidal, square, trapezoidal, triangular waveforms or a combination thereof with:
an amplitude in a range from 1 to 10 mA; and/or
a frequency in a range from 1 to 100 kHz;
pulse width is in a range from 0.01 to 3 ms; and
stimulation is a monopolar, bipolar or tripolar stimulation.

In one embodiment said data about the staging of the cancer of said subject have been obtained by performing a tumor biopsy and/or imaging, preferably by means of magnetic resonance imaging (MRI), computed tomography (CT) and positron emission tomography (PET) scans.

In one embodiment said neural interface (NI) is configured in order to provide an electric stimulus to said target nerve and/or to allow the bidirectional communication between said tumor masses and nerves. In particular, the NI is designed to be tailored according to the dimensions of the target nerve, being placed, or in other words wrapped, around it. According to the dimension of the NI, it can totally enclose the target nerve inside its diameter or leave space between the NI itself and the target nerve. Non-limiting examples of the NI according to the present invention are commercial cuff electrodes. Commercial cuff electrodes are composed of a cylindrical insulating substrate made of polymeric material, e.g. PDMS, polyimide, SU-8, and a conductive metal element, such as Ti, TiPt, Ir, IrOx. Said conductive elements can have a ring shape, a square shape, or any custom irregular shape. The conductive elements are connected to external electronic by means of encapsulated lead wires or custom-designed connectors, such as touch-proof, BNC, SAMTEC, Omnetics. The Nis suitable for the present invention comprise a locking mechanism in order to mechanically stabilize the NI around the nerve, such as suture threads, or custom locking mechanism based on joints. The NI is connected to an implantable stimulator by means of any connection mean known by the skilled person, for example by means of encapsulated lead wires or wireless connection, such as radiofrequency coupling (RF coupling), Bluetooth low energy (BLE) and/or ultrasounds.

In one embodiment, the implantable stimulator can be current-controlled or voltage-controlled delivering pre-defined current pulses to the nerves through the NI, exploiting a capacitive, faradaic, or pseudofaradaic charge injection mechanism.

A “charge injection mechanism” refers to the process of introducing electric charge (either positive or negative) into a material or device.

In one embodiment acquisition and processing of the data relating to the tumor activity is carried out using a software capable of remotely controlling the implantable stimulator via graphic user interface (GUI). By means of a GUI it is possible to acquire data about the staging of the tumor and control the neuromodulation protocol by modifying the neuromodulation parameters. Neural information is gathered from the target nerve through the implanted NI and is analyzed in order to retrieve specifical features able to discriminate the current status of the analyzed tumor. Such information may include, but is not limited to, spike count, evoked compound action potential shape or area, firing rate, and spike shape.

Some non-limiting examples of such cancer that may be treated include cancer of the central nervous system, prostate, pancreas, breast, colon, rectum, skin, liver, ovary, bladder, esophagus, stomach, cervix, bone, urogenital, lung, mesothelial cells and the like.

In one embodiment said neural interface (NI) comprises or consists of a nanostructured material suitable to be used as nanotransductor to convert external energy into electrical potential allowing to modulate the target nerve activity. According to non-limiting examples, such nanostructured materials are metallic nanoparticles, such as gold, silver, platinum and palladium nanoparticles, magnetic nanoparticles, such as magnetite and maghemite, quantum dots, optoelectronic nanostructures, such as porous silicon and hybrid bulk heterojunctions, conductive polymers, such as PEDOT:PSS, Polypyrrole, Polyaniline and poly(thiophene)s and ceramic nanoparticles, such as ZnO, BaTiO3.

Furthermore, the bioelectronic approach does not pose significant technological problems: once the target nerve is identified the implantation procedure is straightforward and could be made under the assistance of several imaging technologies, such as ultrasound or optical microscopy. Implantable or wireless stimulator, as well as nanotransducers that convert external energy (light, ultrasound, electromagnetic field), could also avoid the use of cable, and leads that could potentially drive inflammation, by envisioning a versatile therapeutic approach that could also be practiced outside the hospital, by personalized GUI.

In one embodiment said external energy is near infrared light (NIR) in the range from 700-900 nm, ultrasound, laser, radiofrequency or magnetic field or a combination thereof.

In summary, the Bioelectric approach described by the present invention displays an innovative anticancer approach, with several advantages respect to drug therapies, such as high selectivity, tolerability, and bidirectional control (simultaneous stimulation/recording paradigm). It is worth noting that the Bioelectronic modulation proposed by the present invention is not only to be intended in the traditional way, using electrical force. In this regard, the Nis used for the aims of the present invention will also use other energy sources, such as light or ultrasound, to perform optoelectronic and piezoelectric nerve modulation respectively. This additional embodiment of the present invention allows wireless communication between the energy source and the target nerve, avoiding the use of stimulators and electrical cables, for a safer and less invasive approach to nerve modulation.

In one embodiment, the invention includes combining the disclosed methods of the invention comprising neuromodulatory protocols with one or more neoplastic disease therapeutic and/or palliative therapies. Said neoplastic disease therapeutic and/or palliative therapies can be administered after or before each of the steps of the methods of the present invention. In one embodiment, the present methods are used in combination with the use of one or more anti-cancer agents. As used herein, anti-cancer agents (used interchangeably with “anti-tumor or anti-neoplastic” agent) include any anti-cancer therapies, such as radiation therapy, surgery, hyperthermia or hyperthermia therapy, or anti-cancer compounds useful in the treatment of cancer. These include any agents, when used alone or in combination with other agent, that can alleviate, reduce, ameliorate, prevent, or place or maintain in a state of remission of clinical symptoms or diagnostic markers associated with neoplastic disease, tumors and cancer, and can be used in methods, combinations and compositions provided herein. Exemplary anti-cancer compounds include, but are not limited to, cytokines, chemokines, growth factors, a photosensitizing agents, toxins, anti-cancer antibiotics, chemotherapeutic compounds, radionuclides, angiogenesis inhibitors, signaling modulators, anti-metabolites, anti-cancer vaccines, anti-cancer oligopeptides, mitosis inhibitor proteins, antimitotic oligopeptides, anti-cancer antibodies (e.g., single-chain antibodies), anti-cancer antibiotics, immunotherapeutic agents, bacteria and any combinations thereof. Exemplary cytokines and growth factors include, but are not limited to, interleukins, such as, for example, interleukin-1, interleukin-2, interleukin-6 and interleukin-12, tumor necrosis factors, such as tumor necrosis factor alpha (TNF-α), interferons such as interferon gamma (IFN-γ) granulocyte macrophage colony stimulating factors (GM-CSF), angiogenins, and tissue factors. Photosensitizing agents include, but are not limited to, for example, indocyanine green, toluidine blue, aminolevulinic acid, texaphyrins, benzoporphyrins, phenothiazines, phthalocyanines, porphyrins such as sodium porfimer, chlorins such as tetra(m-hydroxyphenyl)chlorin or tin(IV) chlorin e6, purpurins such as tin ethyl etiopurpurin, purpurinimides, bacteriochlorins, pheophorbides, pyropheophorbides or cationic dyes. Radionuclides, which depending upon the radionuclide, amount and application can be used for diagnosis and/or for treatment. They include, but are not limited to, for example, a compound or molecule containing 11 Carbon, 11 Fluorine, 13Carbon, 15Nitrogen, 18Fluorine, 19Fluorine, 32Phosphate, 60Cobalt, 90Yttrium, 99Technetium, 103Palladium, 106Ruthenium, 111 Indium, 117Lutetium, 125Iodine, 131 Iodine, 137Cesium, 153Samarium, 186Rhenium, 188Rhenium, 192Iridium, 198Gold, 211Astatine, 212Bismuth or 213Bismuth. Toxins include, but are not limited to, chemotherapeutic compounds such as, but not limited to, 5-fluorouridine, calicheamicin, maytansine, double-chain ricin, ricin A chain, abrin, abrin A chain, saporin, modeccin, modeccin A chain, Pseudomonas aeruginosa exotoxin, Cholera toxin, Shigella toxin, E. coli heat labile toxin and Diptheria toxin, doxorubicin, daunomycin, methotrexate, taxol, ricin A, colchicine, cytochasins, monensin, ouabain, mitoxanthrone, vindesine, vinblastine, vincristine and enterotoxin. Anti-metabolites include, but are not limited to, methotrexate, 5-fluorouracil, 6-mercaptopurine, cytosine arabinoside, hydroxyurea and 20-chlorodeoxyadenosine. Signaling modulators include, but are not limited to, for example, inhibitors of macrophage inhibitory factor, toll-like receptor agonists and stat 3 inhibitors. Anti-cancer antibiotics include, but are not limited to, anthracyclines such as doxorubicin hydrochloride (adriamycin), idarubicin hydrochloride, daunorubicin hydrochloride, aclarubicin Hydrochloride, epirubicin hydrochloride and purarubicin hydrochloride, enomycin, phenomycin, pleomycins such as pleomycin and peplomycin sulfate, mitomycins such as mitomycin C, actinomycins such as actinomycin D, zinostatinstimalamer and polypeptides such as neocarzinostatin. Anti-cancer antibodies include, but are not limited to, Rituximab (RITUXAN), ADEPT, Trastuzumab (HERCEPTIN), Tositumomab (BEXXAR), Cetuximab (ERBITUX), Ibritumomab (90Y-lbritumomab tiuexetan; ZEVALIN), Alemtuzumab (Campath-1H), Epratuzumab (Lymphocide), Gemtuzumab ozogamicin (MYLOTARG), Bevacimab (AVASTIN), and Edrecolomab (PANOREX). Angiogenesis inhibitors include, but are not limited to, collagenase inhibitors such as metalloproteinases and tetracyclines such as minocycline, naturally occurring peptides such as endostatin and angiostatin, fungal and bacterial derivatives, such as fumagillin derivatives like TNP-470, aptamer antagonist of VEGF, batimastat, Captopril, cartilage derived inhibitor (CDI), genistein, interleukin 12, Lavendustin A, medroxyprogesterone acetate, recombinant human platelet factor 4 (rPF4), taxol, D-gluco-D-galactan sulfate (Tecogalan(=SP-PG, DS-4152)), thalidomide, thrombospondin. Chemotherapeutic compounds include, but are not limited to platinum; platinum analogs (e.g., platinum coordination complexes) such as cisplatin, carboplatin, oxaliplatin, DWA2114R, NK121, IS 3 295, and 254-S; anthracenediones; vinblastine; alkylating agents such as thiotepa and cyclosphosphamide; alkyl sulfonates such as busulfan, improsulfan and piposulfan; aziridines such as benzodopa, carboquone, meturedopa and uredopa; ethylenimines and methylamelamines including altretamine, triethylenemelamine, trietylenephosphoramide, triethylenethiophosphaoramide and trimethylolomelamime nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; eflornithine; elliptinium acetate; etoglucid; gallium nitrate; substituted ureas; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; anti-cancer polysaccharides; polysaccharide-K; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; cytosine arabinoside; cyclophosphamide; thiotepa; taxoids, such as paclitaxel and doxetaxel; chlorambucil; gemcitabine; 6-thioguanine; mercaptopurine; methotrexate; etoposide (VP-16); ifosfamide; mitomycin C; mitoxantrone; vincristine; vinorelbine; navelbine; novantrone; teniposide; daunomycin; aminopterin; XELODA; ibandronate; CPT11; topoisomerase inhibitor RFS 2000; difluoromethylornithine (DMFO); retinoic acid; esperamicins; capecitabine; methylhydrazine derivatives; Erlotinib (TARCEVA); sunitinib malate (SUTENT); and pharmaceutically acceptable salts, acids or derivatives of any of the above. Also included in this definition are anti-hormonal agents that act to regulate or inhibit hormone action on tumors such as anti-estrogens including, for example, tamoxifen, raloxifene, aromatase inhibiting 4(5)-imidazoles, 4-hydroxytamoxifen, trioxifene, keoxifene, LY117018, onapristone and toremifene (FARESTON); adrenocortical suppressants; and antiandrogens such as flutamide, nilutamide, bicalutamide, leuprolide and goserelin; and pharmaceutically acceptable salts, acids or derivatives of any of the above. Such chemotherapeutic compounds that can be used herein include compounds whose toxicities preclude use of the compound in general systemic chemotherapeutic methods. As used herein, an anti-cancer oligopeptide or an anti-tumor oligopeptide is short polypeptide that has the ability to slow or inhibit tumor growth and/or metastasis. Anti-cancer oligopeptide typically have high affinity for and specificity to tumors enabling them to target tumors. Such oligopeptides include receptor-interacting compounds, inhibitors of protein-protein interactions, enzyme inhibitors, and nucleic acid-interacting compounds. As used herein an antimitotic oligopeptide is an oligopeptide that inhibits cell division. An antimitotic oligopeptide is an exemplary anti-cancer oligopeptide. Exemplary antimitotic oligopeptides include, but are not limited to, tubulysin, phomopsin, hemiasterlin, taltobulin (HTI-286, 3), and cryptophycin.

### Examples

An example of the method proposed in the present invention is illustrated with reference to FIG. 1 and may be schematized with the workflow according to FIG. 2:

Once the patient has performed tumor biopsy and imaging (MRI, CT and PET scans), a staging of cancer spreading is possible and is aimed to identify the exact location of the primary tumor mass and of the number and position of the metastatic masses. This step is crucial to assess which organs are involved in the process of tumor mass invasion.

Once the invaded organs have been characterized, the identification of tumor innervation will be conducted. This step will be carried out by integrating available anatomical data regarding organ innervation by autonomic and sensory nerve with our previous investigation on cadaveric cancer tissue samples. An alternative solution could be represented by the use of MR neurography, a novel imaging technique able to identify small nerves such the vagus nerve or thoracic nerves. This will allow us to identify the nature of the fibers that innervate the TME as well as the path they follow to reach the TME starting from the anatomically known trunks that innervate the target organs of the tumor. This investigation will be carried out for all the tumor masses (primary and metastatic lesions) previously identified by biopsy and imaging. This step is also important to define the best Neural Interface design and Neuromodulation protocol (stimulatory of inhibitory), both defined according to the nature and geometry of the TME innervation profile.

Once TME innervation has been characterized for the primary and metastatic tumor sites, a dedicated neural interface for each tumor mass to treat will be implanted on the target nerve that innervate the TME. This step will be carried out by neurosurgeons using image guidance to identify the target nerve branch. Moreover, implantable stimulators will also be placed nearby the electrode to ensure appropriate current delivery to each neural electrode implanted. Once each neural interface is implanted another electrode will be temporarily placed downstream to it, in order to record action potential propagation activated by the first implanted electrode. This step is carried out during surgery and is important to ensure that the neural interface is effectively working and can modulate the activity of the target nerve. Prior to start the neuromodulation protocol, the electrodes are monitored for few days to allow their integration within the patient's tissue and assess whether the foreign body reaction to the device caused a consistent inflammatory process. In this case of, an anti-inflammatory drug will be locally delivered.

After neural interface implantation and testing the neuromodulation protocol is started. The parameters of neuromodulation (amplitude, frequency, pulse width, duration of single stimulation, number of daily sessions) will be decided based on the tumor evolution kinetic, available in literature and based on the result of the tumor staging.

For neural stimulation the following set of parameters will be considered:
Amplitude 1-10 mA.
Frequency 1-100 Hz.
Pulse width 0.01-3 ms.
Mono/bipolar stimulation.

For neural inhibition the following set of parameters will be considered:
Amplitude 1-10 mA.
Frequency 1-100 kHz.
Pulse width 0.01-3 ms.
Mono/bipolar stimulation.

In principle for indolent tumor growth fewer daily sessions will be used respect to aggressive tumors. A more precise tuning of neuromodulation parameters will be operated by recording action potential within the target nerve where the neural interface is implanted. Nerves establish a bidirectional communication within the brain and the TME, with afferent fibers used by the TME to send information to the brain according to its activity and efferent fibers carrying information used to modulate the TME properties in a pro-tumoral fashion. While modulating the neurotransmitters release within the TME, a variation of the electrical activity of the target nerve is expected. This variation will be used to calibrate the neuromodulation protocol during time. This step is fundamental, because the establishment of a closed-loop control between the TME innervation and the neural interface and will be used not only to control the neuromodulation, but also to acquire information on tumor activity, crucial to tune the nerve activity to reduce tumor growth.

A fine and real-time tuning of neuromodulation parameter will be possible with specific software that can remotely control the implantable stimulator using GUIs. These apps will be used by the physician and the engineers to acquire information on the bioelectronic therapy, to vary its parameters and possibly to force a block in the event of electrical malfunctions of the electrode which can alter the therapy or produce annoying electric shocks for the patient.

Periodic staging of tumor spreading will be also performed using PET and CT scans, integrating the acquired information on tumor activity provided by the neural interface with morphological/functional data on tumor mass localization, spreading and metabolic activity. After a certain period (i.e. 20 days), tumor staging will reveal whether the bioelectronic therapy succeeded to reduce cancer growth. In this regard, two possible scenarios may arise:
1. The tumor masses or the metabolic activity did not respond to the therapy: in this case we firstly continue with the therapy until the next periodic staging changing the neuromodulation parameters and
2. The tumor masses or the metabolic activity responded to the therapy: in this case we simply continue to modulate the activity of the target nerve without changing the neuromodulation parameters until the next periodic staging. If one of the treated metastases or even the main tumor mass disappears, the neural interface connected to that mass would be removed through surgery.

The neuromodulation protocol will continue until patient remission or until the next periodic staging.

This neuromodulation protocol is thought to be run in association with current chemotherapeutic protocols, to enhance their efficacy and possibly reduce their systemic side effects, by reducing the administered dose.

1. A method for the treatment of a cancer in a subject, or for adjuvating the treatment of cancer in a subject comprising the following steps:
i) implanting a neural interface (NI) on at least one target nerve of the tumor microenvironment (TME) of a tumor mass of said cancer, and at least one stimulator connected to said NI in subject in need of such treatment; and
ii) performing a neuromodulation protocol to said nerve using said neural interface in order to reduce the tumor mass or the metabolic activity of said cancer.

2. The method according to claim 1, wherein said neuromodulation protocol consists of stimulating or inhibiting said target nerve.

3. The method according to claim 1, wherein said stimulator is implanted in a subcutaneous cavity.

4. The method according to claim 1, wherein said target nerve is determined by characterization of the parasympathetic, sympathetic and/or sensory innervation of the tumor microenvironment (TME) for primary and metastatic tumor masses of said cancer.

5. The method of claim 4, wherein said target nerve is determined by combining imaging information with physiological and/or neural information.

6. The method for according to claim 1, further comprising prior to step i) the following steps:
a) acquiring data about the staging of the cancer of said subject;
b) defining the tumor microenvironment (TME) innervation profile of each tumor mass of said cancer;

7. A method for the treatment of a cancer in a subject, or for adjuvating the treatment of cancer in a subject, comprising the following steps:
i) acquiring data about the staging of the cancer of said subject before the treatment;
ii) defining the tumor microenvironment (TME) innervation profile of each tumor mass of said cancer;
iii) implanting a neural interface (NI) on at least one target nerve of the tumor microenvironment (TME) of a tumor mass of said cancer, and at least one stimulator connected to said NI;
iv) performing a neuromodulation protocol using said neural interface with neuromodulation parameters related to the tumor staging before the treatment;
v) comparing data about the staging of the cancer of said subject before and after the treatment; and
vi): a) if no reduction of said tumor mass or the metabolic activity has been achieved, repeating the step iv) changing the neuromodulation parameters; or
b) if reduction of said tumor mass or the metabolic activity has been achieved, repeating step iv) without changing the neuromodulation parameters.

8. A method for the treatment of a cancer in a subject, or for adjuvating the treatment of cancer in a subject, comprising the following steps:
i) acquiring data about the staging of the cancer of said subject before the treatment;
ii) defining the tumor microenvironment (TME) innervation profile of each tumor mass of said cancer;
iii) implanting a neural interface (NI) on all the nerves of the tumor microenvironment (TME) of each tumor mass of said cancer, and their stimulators connected to said NI; and
iv) performing a neuromodulation protocol using said neural interface in order to reduce the tumor mass or the metabolic activity of said cancer.

9. The method according to claim 1, wherein said metabolic activity is the glucose consumption of said cancer, as measured by positron emission tomography (PET).

10. The method according to claim 1, wherein said neuromodulation protocol uses the following parameters for stimulating said target nerve:
biphasic rectangular, sinusoidal, trapezoidal, triangular pulses or a combination thereof, with:
an amplitude in a range from 1 to 10 mA; and/or
a frequency in a range from 1 to 100 Hz;
pulse width is in a range from 0.01 to 3 ms; and
stimulation is a monopolar, bipolar or tripolar stimulation.

11. The method according to claim 1, wherein said neuromodulation protocol uses the following parameters for inhibiting said target nerve:
biphasic sinusoidal, square, trapezoidal, triangular waveforms or a combination thereof with:
an amplitude in a range from 1 to 10 mA; and/or
a frequency in a range from 1 to 100 kHz;
pulse width is in a range from 0.01 to 3 ms; and
stimulation is a monopolar, bipolar or tripolar stimulation.

12. The method according to claim 6, wherein said data about the staging of the cancer of said subject have been obtained by performing a tumor biopsy and/or imaging, optionally by means of magnetic resonance imaging (MRI), computed tomography (CT) and positron emission tomography (PET) scans.

13. The method according to claim 1, wherein said neural interface (NI) is configured in order to provide an electric stimulus to said target nerve and/or to allow the bidirectional communication between said tumor masses and nerves.

14. The method according to claim 1, wherein said neural interface (NI) is placed around the target nerve.

15. The method according to claim 1, wherein said neural interface (NI) is connected to said stimulator by means of encapsulated lead wires or wireless connection, and optionally radiofrequency coupling (RF coupling), bluetooth low energy (BLE) and/or ultrasounds.

16. The method according to claim 1, wherein said stimulator is current-controlled and delivers current pulses to said target nerve through the neural interface (NI) exploiting a capacitive, faradaic or pseudo faradaic charge injection mechanism.

17. The method according to claim 1, wherein said neural interface (NI) comprises or consists of a nanostructured material suitable to be used as nano transductor to convert external energy into electrical potential, optionally wherein said external energy is near infrared light (NIR) in the range from 700 to 900 nm, ultrasound, laser, radiofrequency, magnetic field or a combination thereof.

18. The method according to claim 6, wherein said data relating to the staging of the tumor are selected from spike count, evoked compound action potential shape or area, firing rate and spike shape.

19. The method according to claim 1, wherein said cancer is selected from the group consisting of central nervous system cancer, pancreatic cancer, prostate cancer, breast cancer, colon cancer, rectum cancer, skin cancer, liver cancer, cervical cancer, lung cancer, ovarian cancer, bladder cancer, bone cancer, stomach cancer, urogenital cancer, esophageal cancer, mesothelial cells and combinations thereof.

20. The method according to claim 1, further comprising a step of administering one or more neoplastic disease therapeutic and/or palliative therapies, optionally comprising the administration of one or more anti-cancer agent.

21. The method according to claim 20, wherein said one or more anti-cancer agent is selected from the group consisting of cytokines, chemokines, growth factors, a photosensitizing agents, toxins, anti-cancer antibiotics, chemotherapeutic compounds, radionuclides, angiogenesis inhibitors, signaling modulators, anti-metabolites, anti-cancer vaccines, anti-cancer oligopeptides, mitosis inhibitor proteins, antimitotic oligopeptides, anti-cancer antibodies (e.g., single-chain antibodies), anti-cancer antibiotics, immunotherapeutic agents, bacteria and combinations thereof.
